(19) United States
(12) Patent Application Publication
(10) Pub. No.: US 2024/0058387 A1
(43) Pub. Date: Feb. 22, 2024

(54) CULTURE MEDIUM COMPOSITION FOR AMPLIFYING AND MAINTAINING SELF-RENEWAL CAPACITY AND DIFFERENTIATION POTENTIAL OF HSCS AND APPLICATION THEREOF

(71) Applicants: EDIGENE (GUANGZHOU) INC., Guangzhou City, Guangdong (CN); EDIGENE INC., Beijing (CN)

(72) Inventors: Riguo FANG, Beijing (CN); Zhongyu SHI, Beijing (CN); Huihui YANG, Beijing (CN); Pengfei YUAN, Beijing (CN)

(21) Appl. No.: 18/270,181

(22) PCT Filed: Dec. 28, 2021
(86) PCT No.: PCT/CN2021/142127

§ 371 Date: Jun. 28, 2023

(30) Foreign Application Priority Data
Dec. 28, 2020 (WO) PCT/CN2020/140172

(51) Int. Cl. A61K 35/28; C12N 5/00; C12N 5/0789; A61P 7/06

(52) U.S. Cl. CPC A61K 35/28; C12N 5/0018; C12N 5/0647; A61P 7/06

(57) ABSTRACT

A culture medium composition for expanding hematopoietic stem cells (HSCs) and maintaining self-renewal capacity and differentiation potential of HSCs, a cell population and an application thereof. The culture medium composition comprises a hematopoietic stem cell medium and a small molecule inhibitor of a PDGFR target. The inhibitor of PDGFR can significantly expand HSCs during in-vitro culture, maintaining a high proportion of HSCs with self-renewal capacity, achieving in-vitro expansion of HSCs while maintaining a relatively high proportion of cells with sternness.

### TECHNICAL FIELD

The present application relates to the technical field of biotechnology, in particular to a culture medium composition for expanding HSCs and maintaining the self-renewal ability and differentiation potential of HSCs, an infusion solution containing the HSCs, and applications thereof.

### BACKGROUND TECHNIQUE

Hematopoietic stem cells (HSCs) are a group of heterogeneous primitive hematopoietic cells in the blood system, with two important characteristics of self-renewal and multi-lineage differentiation. When the body is in a healthy state, the HSCs in the body are in a quiescent state for a long time. When the body is in a state of disease or severe blood loss, the HSCs are activated and enter a state of self-renewal and multi-lineage differentiation to maintain the stability of the blood system and the body's homeostasis. The self-renewal properties of HSCs are beneficial to the maintenance of stemness of progeny HSCs, while the multi-lineage differentiation properties of HSCs allow them to differentiate into a variety of mature blood cells, such as myeloid cells (granulocytes, monocytes, erythrocytes and platelets), and lymphoid cells (T cells and B cells). Because of the characteristics of HSCs and their ability to migrate and homing in the blood system, it is beneficial for HSCs to differentiate when the body needs them, and to homing to the bone marrow microenvironment to function when the body is in homeostasis.

These properties of HSCs make it possible to treat hematological diseases by hematopoietic stem cell transplantation (HSCT). In 1959, Thomas et al. used bone marrow hematopoietic stem cells for the first hematopoietic stem cell transplantation in human history to treat leukemia in clinical to restore normal hematopoietic function in patients. In the following decades, through the continuous efforts of scientific researchers, hematopoietic stem cell transplantation has not only been used to treat a variety of blood system diseases, but also used to treat immunodeficiency diseases and neurodegenerative diseases and so on.

At present, there are three main sources of HSCs, bone marrow (BM), mobilized peripheral blood (mPB), and umbilical cord blood (CB). The three sources of HSCs have their own advantages and disadvantages, for example, the collection of bone marrow-derived hematopoietic stem cells is invasive and insufficient; the proportion of HSCs in human peripheral blood is too low (less than 0.1%), and during collection, granulocyte colony-stimulating factor (G-CSF) is required to mobilize hematopoietic stem cells to migrate from bone marrow to peripheral blood, and in clinical applications, the mobilization effect is poor, the number of HSCs is insufficient, resulting in repeated mobilization or transplantation failure; collection of hematopoietic stem cells from umbilical cord blood is convenient, harmless to the donor and has no ethical issues, and the collected HSCs have strong hematopoietic ability. Among the above three sources of HSCs, HSCs derived from bone marrow and mobilized peripheral blood all require human leukocyte antigen (HLA) matching between donors and patients. HLA matching is difficult. Once mismatch occurs, graft versus host disease (GVHD) will occur, and a large number of patients with GVHD will die of immune system disorders. HSCs derived from umbilical cord blood have low requirements for the degree of HLA matching, allowing partial HLA mismatch, and the incidence of GVHD after transplantation is low, which alleviates the difficulty of traditional HSCT matching. The common problem faced by the HSCs collected by the above three methods is the small amount of cells, which sometimes can only meet the transplantation needs of children or light-weight adults, but cannot meet the transplantation needs of larger-weight adults.

Studies have shown that the safety and efficacy of hematopoietic stem cell transplantation depend on the content of transplanted HSCs. When the number of transplanted HSCs is insufficient, the recovery of neutrophils in patients is delayed, resulting in an increased risk of GVHD. The higher the content of transplanted HSCs, the shorter the recovery time of neutrophils and platelets, and the shorter hospital care time, which greatly reduces the risk of transplant failure and reduces the burden on patients.

The characteristics of self-renewal and multi-lineage differentiation of hematopoietic stem cells lead to the differentiation of hematopoietic stem cells into blood cells of various lineages and the loss of self-renewal characteristics once they divide and proliferate in large numbers in the process of in vitro culture. Therefore, researchers continue to make efforts to explore the use of different methods to expand hematopoietic stem cells in vitro to a certain extent, while maintaining the self-renewal capacity of hematopoietic stem cells to the maximum extent. If it can be realized, the success rate of hematopoietic stem cell transplantation can be improved. So far, one of the ideas for culturing hematopoietic stem cells in vitro is to add small molecule compounds to the medium to target and regulate the division and proliferation signals of hematopoietic stem cells, so that hematopoietic stem cells can maintain a certain degree of expansion while sustaining its self-renewal capacity by changing the state of cell division and proliferation.

Studies have shown that platelet-derived growth factor PDGF (platelet-derived growth factor) and platelet-derived growth factor receptor PDGFR (platelet-derived growth factor receptor) are closely related to cell division and proliferation. PDGF is a pro-angiogenic factor isolated from human platelets, and PDGFR is a member of the tyrosine protein kinase family located on the cell membrane. Studies have shown that PDGF must bind to PDGFR, activating PDGFR by phosphorylation, and starting the PDGF/PDGFR signaling pathway in order to exert biological effects, such as stimulating fibroblasts, glial cells, smooth muscle cells, etc. arrested at G0/G1 phase to enter the division and proliferation cycle. When the body is damaged, a large amount of PDGF released by platelets can stimulate the proliferation of adjacent connective tissue cells, thereby rebuilding damaged tissues and healing wounds. This signaling pathway has also been confirmed to be associated with the occurrence and development of a series of diseases. In a variety of tumors, the expression of PDGF and PDGFR is closely related to the occurrence and development of tumors. Tumor cells promote angiogenesis by releasing PDGF, induce tumor cell proliferation and migration, and inhibit their apoptosis. According to the action mechanism of PDGF/PDGFR, great progress has been made in the targeted therapy of tumors, and a number of inhibitor drugs against PDGFR have been approved for marketing. The PDGF/PDGFR signaling pathway has been widely reported in many types of cells, but less reported in hematopoietic stem cells. The role of PDGF/PDGFR signaling pathway in hematopoietic stem cell expansion and maintenance of self-renewal capacity is still a research gap.

### SUMMARY OF THE APPLICATION

In view of the above problems, the present application provides a culture medium composition for expanding HSCs and maintaining the self-renewal ability and differentiation potential of HSCs, a cell population and an application thereof.

When the inventors of the present application culture HSCs from different sources in vitro, by continuously adding PDGFR inhibitors the expansion of HSCs can be maintained to a certain extent, while at the same time the self-renewal ability of HSCs is maintained at a high proportion, so that in the cell culture product, a large number of LT-HSCs with transplantation potential can be obtained, and the effect is better than that of known chemical small molecules for culturing HSCs. This is the first report in the study of expansion and self-renewal capacity of hematopoietic stem cells.

The specific technical scheme of the present application is as follows:
1. A culture medium composition for expanding hematopoietic stem cells (HSCs) and maintaining self-renewal ability and differentiation potential of HSCs, comprising a hematopoietic stem cell medium and a small molecule inhibitor of a PDGFR target.
2. The culture medium composition according to item 1, wherein the small molecule inhibitor of a PDGFR target is one or more selected from the group consisting of: AG1296, PDGFR inhibitor 1, Imatinib, PP121, Ponatinib, Axitinib, Trapidil and Erdafitinib, preferably AG1296.
3. The culture medium composition according to item 1 or 2, wherein the hematopoietic stem cell medium comprises: 1) a basal medium (preferably a serum-free basal medium); 2) a growth factor; and/or 3) a cytokine.
4. The culture medium composition according to item 3, wherein the growth factor or cytokine is one or more selected from the group consisting of: growth factor Flt-3L, growth factor SCF, growth factor TPO and interleukin IL-6.
5. The culture medium composition according to item 4, wherein the concentrations of the growth factors or cytokines in the culture medium composition are as follows:
the concentration of the growth factor Flt-3L is 10-110 ng/ml, preferably 50-100 ng/ml;
the concentration of the growth factor SCF is 10-110 ng/ml, preferably 50-100 ng/ml;
the concentration of the growth factor TPO is 10-110 ng/ml, preferably 50-100 ng/ml; and
the concentration of the interleukin IL-6 is 1-50 ng/ml, preferably 1-20 ng/ml. 6. The culture medium composition according to any one of items 1-5, wherein the concentration of the small molecule inhibitor of a PDGFR target in the culture medium composition is 0.1-100 μM, preferably 0.5-50 μM, more preferably 1-10 μM.
7. The culture medium composition according to any one of items 1-6, wherein the HSCs are derived from bone marrow, mobilized peripheral blood, umbilical cord blood, cryopreserved and resuscitated HSCs or HSCs modified by gene editing.
8. A method for promoting the expansion of HSCs and maintaining the self-renewal capacity of the HSCs, comprising in vitro culturing the HSCs in a culture medium composition containing a small molecule inhibitor of a PDGFR target.
9. The method according to item 8, wherein the small molecule inhibitor of a PDGFR target is one or more selected from the group consisting of: AG1296, PDGFR inhibitor 1, Imatinib, PP121, Ponatinib, Axitinib, Trapidil and Erdafitinib, preferably AG1296.
10. The method according to item 8 or 9, wherein the hematopoietic stem cell medium comprises: 1) a basal medium (preferably a serum-free basal medium); 2) a growth factor; and/or 3) a cytokine.
11. The method according to item 10, wherein the growth factor or cytokine is one or more selected from the group consisting of: growth factor Flt-3L, growth factor SCF, growth factor TPO and interleukin IL-6.
12. The method according to item 11, wherein the concentrations of the growth factors or cytokins in the culture medium composition are as follows:
the concentration of the growth factor Flt-3L is 10-110 ng/ml, preferably 50-100 ng/ml;
the concentration of the growth factor SCF is 10-110 ng/ml, preferably 50-100 ng/ml;
the concentration of the growth factor TPO is 10-110 ng/ml, preferably 50-100 ng/ml; and
the concentration of the interleukin IL-6 is 1-50 ng/ml, preferably 1-20 ng/ml.
13. The method according to any one of items 8-12, wherein the concentration of the small molecule inhibitor of a PDGFR target in the culture medium composition is 0.1-100 μM, preferably 0.5-50 μM, more preferably 1-10 jam.
14. The method according to any one of items 8-13, wherein the HSCs are derived from bone marrow, mobilized peripheral blood, umbilical cord blood, cryopreserved and resuscitated HSCs or HSCs modified by gene editing.
15. The method according to any one of items 8-14, wherein the in vitro culture time is about 4-21 days, preferably about 6-15 days, more preferably about 6-10 days, most preferably about 6-8 days.

16. The method according to any one of items 8-15, wherein after in vitro culture, the cell number of CD34+ phenotype HSCs accounts for 40-85% of the total cells, preferably 60-85%, more preferably 75%-80%.
17. The method according to any one of items 8-16, wherein after in vitro culture, the cell number of CD34+CD90+ phenotype HSCs accounts for 6-15% of the total cells, preferably 8-15%, more preferably 8-12%.
18. The method according to any one of items 8-17, wherein after in vitro culture, the cell number of CD34+CD90+CD45RA− phenotype HSCs accounts for 2-10% of the total cells, preferably 2-6%, more preferably 4-5%.
19. The method according to any one of items 8-18, wherein after in vitro culture, the cell number of CD34+CD45+CD90+CD45RA−CD38− phenotype HSCs accounts for 2-5% of the total cells, preferably 2.5-4%.
20. An HSCs infusion solution, wherein the cell number of CD34+ phenotype HSCs accounts for 40-85% of the total cells, preferably 60-85%, more preferably 75-80%.
21. The HSCs infusion solution according to item 20, wherein the cell number of CD34+CD90+ phenotype HSCs accounts for 6-15% of the total cells, preferably 8-15%, more preferably 8-12%.
22. The HSCs infusion solution according to item 20 or 21, wherein the cell number of CD34+CD90+CD45RA− phenotype HSCs accounts for 2-10% of the total cells, preferably 2-6%, more preferably 4-5%.
23. The HSCs infusion solution according to any one of items 20-22, wherein the cell number of CD34+CD45+CD90+CD45RA−CD38− phenotype HSCs accounts for 2-5% of the total cells, preferably 2.5-4%.
24. The HSCs infusion solution according to any one of items 20-23, which is obtained by the method of any one of items 8-20.
25. A method for replenishing blood cells to an individual in need, comprising infusing the HSCs infusion solution of any one of items 20-24 to the individual.
26. The method according to item 25, wherein after the HSCs infusion solution is infused into the individual, the HSCs colonize and differentiate into blood cells in the individual.
27. The method according to item 25 or 26, wherein the individual is an individual suffering from hemorrhage, anemia, cancer, leukemia, autoimmune disease, viral or bacterial infection.
28. Use of a small molecule inhibitor of a PDGFR target in promoting the expansion of HSCs and maintaining the self-renewal ability of HSCs, preferably, the small molecule inhibitor of a PDGFR target is one or more selected from the group consisting of: AG1296, PDGFR inhibitor 1, Imatinib, PP121, Ponatinib, Axitinib, Trapidil and Erdafitinib, preferably AG1296.
29. A method for preventing or treating a disease in an individual, comprising infusing the HSCs infusion solution of any one of items 20-24 to the individual.
30. Use of the HSCs infusion solution according to any one of items 20-24 in the preparation of a medicament for preventing or treating a disease.
31. The use according to item 30, wherein the disease is a disease requiring replenishment of blood cells.

### Effect of the Application

The applicant's research results demonstrate for the first time that inhibitors of PDGFR can significantly expand HSCs during in vitro culture, while maintaining a high proportion of HSCs' self-renewal capacity. The PDGFR inhibitor discovered by the applicant is significantly more effective than the reported small chemical molecules in expanding LT-HSCs. This is the first time to demonstrate and report that the PDGF/PDGFR signaling pathway plays an important role in the expansion of hematopoietic stem cells and the maintenance of self-renewal capacity. The applicant's research results can realize the in vitro expansion of HSCs while maintaining sternness of a relatively high proportion of HSCs. On this basis, the clinical application of HSCs transplantation can widely treat a series of blood system diseases.

### BRIEF DESCRIPTION OF THE DRAWINGS

FIG. 1 shows the determination of the logic gate and gate position of the target cell population CD34+CD45+CD45RA−CD90+CD38− cell population.

FIG. 2 shows the screening of optimal concentration of the compounds and maintaining the sternness of HSCs on cord blood-derived CD34+ cells. The expression analysis diagram of LT-HSCs cell surface markers (CD34+CD45+CD90+CD45RA−CD38−) detected by flow cytometry after induced by the compounds in Table 1 (3 test concentrations of each compound) for 6-8 days is shown. The abscissa represents the name of the inhibitor and the concentration used, and the ordinate represents the expansion fold of the ratio of LT-HSCs in the text group/control group.

FIG. 3 3A shows the screening of optimal concentration of the compound AG1296 to maintain the sternness of HSCs on umbilical cord blood-derived CD34+ cells. The expression analysis diagram of the cell surface markers of LT-HSCs (CD34+CD45+CD90+CD45RA−CD38−) detected by flow cytometry after induced by the compound for 6 days is shown. The abscissa represents the name of the inhibitor and the concentration used, the ordinate CD34+(%), CD34+CD90+(%), CD34+CD90+CD45RA−(%), CD34+CD45+CD90+CD45RA− CD38−(%) represent the proportion of cells expressing different markers in total cells.

3B shows the screening of optimal concentration of the small molecule compound AG1296 to expand HSCs on cord blood-derived CD34+ cells. The total cell number is counted after 6 days of compound induction treatment, the expression of cell surface markers (CD34+CD45+CD90+CD45RA−CD38−) are detected by flow cytometry, and the expression analysis diagram is shown. And the absolute number of CD34+ cells, CD34+CD90+ cells, CD34+CD90+CD45RA− cells, CD34+CD90+CD45RA−CD38− cells proliferation is obtained according to the cell count results, wherein the abscissa represents the name and concentration of the inhibitor, the number of cells (*e5) on the ordinate represents the absolute number of cells (absolute number of cells=total number of cells*sternness ratio, wherein the sternness ratio refers to the ratio of the cells screened by using the combination of hematopoietic stem cell surface marker molecules).

FIG. 4 4A shows the screening of optimal concentration of the compound AG1296 to maintain the sternness of HSCs on umbilical cord blood-derived CD34+ cells. The expression analysis diagram of cell surface markers of LT-HSCs (CD34+CD45+CD90+CD45RA− CD38−) detected by flow cytometry after induced by the compound for 6 days is shown. The abscissa represents the name of the inhibitor and the concentration used, the ordinate CD34+(%), CD34+CD90+(%), CD34+CD90+CD45RA−(%), CD34+CD45+CD90+CD45RA− CD38−(%) represent the proportion of cells expressing different markers in total cells.

4B shows the screening of optimal concentration of the small molecule compound AG1296 to expand HSCs on cord blood-derived CD34+ cells. The total cell number is counted after 6 days of compound induction treatment, and the cell surface markers (CD34+CD45+CD90+CD45RA−CD38−) are detected by flow cytometry, and the expression analysis diagram is shown. And the absolute number of CD34+ cells, CD34+CD90+ cells, CD34+CD90+CD45RA− cells, CD34+CD90+CD45RA−CD38− cells proliferation is obtained according to the cell count results, wherein the abscissa represents the name and concentration of the inhibitor, the number of cells (*e5) on the ordinate represents the absolute number of cells (absolute number of cells=total number of cells*sternness ratio, where the sternness ratio refers to the ratio of the cells screened by using the combination of hematopoietic stem cell surface marker molecules).

FIG. 5 5A shows the comparison of compound AG1296 with known literature-reported inhibitors SR1 and UM171 in maintaining sternness of HSCs on mobilized peripheral blood-derived CD34+ cells. The expression analysis diagram of LT-HSCs cell surface markers (CD34+CD45+CD90+CD45RA−CD38−) detected by flow cytometry after induced by the compounds for 8 days is shown. The abscissa represents the name of the inhibitor and the concentration used, the ordinate CD34+(%), CD34+CD90+(%), CD34+CD90+CD45RA−(%), CD34+CD45+CD90+CD45RA−CD38−(%) represent the proportion of cells expressing different markers in total cells.

5B shows the comparison of compound AG1296 with known literature-reported inhibitors SR1 and UM171 in expanding cells on mobilized peripheral blood-derived CD34+ cells. The expression analysis diagram of cell surface markers (CD34+CD45+CD90+CD45RA−CD38−) detected by flow cytometry after compound induction treatment for 8 days is shown. And the absolute number of CD34+ cells, CD34+CD90+ cells, CD34+CD90+CD45RA-cells, and CD34+CD90+CD45RA−CD38− cells proliferation is obtained according to the cell count results, wherein the abscissa represents the name of the inhibitor, and the ordinate number of cells (*e5) represents the absolute number of cells (absolute number of cells=total number of cells*sternness ratio, wherein the sternness ratio refers to the ratio of the cells screened by using the combination of hematopoietic stem cell surface marker molecules).

FIG. 6 shows an analysis graph of the in vitro clonogenic ability of cord blood-derived CD34+ cells with various concentrations of AG1296. BFU-E, CFU-E, CFU-GM, CFU-GEMM represent clones of different lineages of blood system such as erythroid, myeloid, and lymphoid. Among them, the abscissa represents the name of the inhibitor and the concentration used, the number of clones on the ordinate represents the total number of clones, and the number of GEMM clones represents the number of CFU-GEMM clones.

FIG. 7 shows the determination of logic gates and gate positions for the hCD45+, hCD19+, hCD33+, hCD3+ and hCD56+ cell populations of interest.

FIGS. 8 8A show the comparison of the effect of compound AG1296 and the known inhibitor SR1 reported in the literature on the in vivo transplantation of the in vitro cultured and mobilized peripheral blood-derived CD34+ cells in immunodeficient mice. The CD34+ cells derived from mobilized peripheral blood were cultured with small molecule inhibitors in vitro for 6 days, and transplanted to immunodeficiency mice. The proportion of human CD45+ cells in the bone marrow cells of the mice is detected 18 weeks after transplantation. The abscissa represents the name of the inhibitor, and the ordinate represents the proportion of human CD45+ cells in the mouse bone marrow cells.

8B shows the comparison of the effect of compound AG1296 and the known inhibitor SR1 reported in the literature on the ability of the in vitro cultured and mobilized peripheral blood-derived CD34+ cells to form cells of each lineage after transplantation in immunodeficient mice in vivo. The CD34+ cells derived from mobilized peripheral blood were cultured with small molecule inhibitors in vitro for 6 days, and then transplanted in immunodeficient mice. 18 weeks after the transplantation, the proportions of human CD19+(representing B cells), human CD33+(representing Myeloid cells), human CD3+(representing T cells) and human CD56+(representing NK cells) cells in the mouse bone marrow cells are detected. The abscissa represents the name of the inhibitor, and the ordinate represents the proportion of human lineage cells in mouse bone marrow cells.

### DETAIL DESCRIPTION OF THE APPLICATION

The present application will be described in detail below with reference to the embodiments described in the drawings, wherein like numerals represent like features throughout the drawings. Although specific embodiments of the present application are shown in the drawings, it should be understood that the present application may be embodied in various forms and should not be limited by the embodiments set forth herein. Rather, these embodiments are provided so that there will be a more thorough understanding of the present application, and will fully convey the scope of the present application to those skilled in the art.

It should be noted that certain terms are used in the description and claims to refer to specific components. It should be understood by those skilled in the art that the same component may be referred to by different nouns. The present specification and claims do not take the difference in terms as a way to distinguish components, but take the difference in function of the components as a criterion for distinguishing. As referred to throughout the specification and claims, “comprising” or “including” are open-ended terms and should be interpreted as “including but not limited to”. Subsequent descriptions in the specification are preferred embodiments for implementing the present application, however, the descriptions are for the purpose of general principles of the specification and are not intended to limit the scope of the present application. The scope of protection of the present application should be determined by the appended claims.

The present application provides a culture medium composition for expanding HSCs and maintaining the self-renewal ability and differentiation potential of HSCs, which comprises a small molecule inhibitor of a PDGFR target.

The self-renewal ability of HSCs refers to the ability to generate HSCs with sternness without differentiation.

The small molecule inhibitor refers to a molecular entity (usually organic or organometallic) that is not a polymer, has pharmaceutically activity, and has a molecular weight of less than about 2 kDa, less than about 1 kDa, less than about 900 Da, less than about 800 Da, or less than about 700 Da. The small molecule inhibitors can be synthetic, semi-synthetic (ie, synthesized from naturally occurring precursors) or obtained by biological means.

In a preferred embodiment of the present application, the small molecule inhibitor of a PDGFR target is one or more selected from the group consisting of: AG1296, PDGFR inhibitor 1, Imatinib, PP121, Ponatinib, Axitinib, Trapidil and Erdafitinib; preferably AG1296.

The AG1296 is a synthetic quinoline compound, which is an enzyme inhibitor, and can compete with ATP to inhibit PDFGR.

The PDGFR inhibitor 1 is a synthetic enzyme inhibitor that can inhibit the PDGFR target.

The Imatinib is a synthetic multi-target tyrosine kinase inhibitor, which can inhibit the PDGFR target.

The PP121 is a synthetic multi-target inhibitor, which can inhibit the PDGFR target.

The Ponatinib is a synthetic multi-target inhibitor, which can inhibit the PDGFR target.

The Axitinib is a synthetic multi-target inhibitor, which can inhibit the PDGFR target.

The Trapidil is a synthetic PDGF antagonist that can disrupt the autocrine loops of PDGF and PDGFR.

The Erdafitinib is a synthetic FGFR inhibitor that can also inhibit a PDGFR target.

In a preferred embodiment of the present application, wherein the composition further comprises a hematopoietic stem cell medium, preferably, the hematopoietic stem cell medium comprises: 1) a basal medium (preferably a serum-free basal medium) 2) a growth factor; and/or 3) a cytokine; The growth factor or cytokine is one or more selected from the group consisting of: growth factor Flt-3L, growth factor SCF, growth factor TPO and interleukin IL-6;

Preferably, the concentration of the growth factor Flt-3L is 10-110 ng/ml, preferably 50-100 ng/ml;

The concentration of the growth factor SCF is 10-110 ng/ml, preferably 50-100 ng/ml;

The concentration of the growth factor TPO is 10-110 ng/ml, preferably 50-100 ng/ml; and

The concentration of the interleukin IL-6 is 1-50 ng/ml, preferably 1-20 ng/ml.

The basal medium refers to a medium that can provide basal nutritive substances required for cell proliferation, all basal mediums comprise amino acids, vitamins, carbohydrates, inorganic ions and other substances, and the basal medium can be self-made (i.e. it is required to use powder to make liquid by oneself), or it can be commercially available (ie, liquid). The basal medium includes serum-containing basal medium and serum-free basal medium.

The serum in the serum-containing basal medium can be fetal bovine serum or calf serum, etc.

The serum-free basal medium can be, for example, SFEM, SFEM II, StemSpan® H3000, and StemSpan′ ACF from StemCell Company;

StemPro-34 from ThermoFisher Company; Stemline II from Sigma Company; StemXVivo from R&D Company; X-VIVO 15 from Lonza Company; and SCGM from CellGenix Company, etc.

The growth factor Flt-3L refers to human FMS-related tyrosine kinase 3 ligand, which can stimulate the proliferation of hematopoietic stem cells.

The growth factor SCF refers to human stem cell factor, which can stimulate the proliferation of hematopoietic stem cells.

The growth factor TPO refers to human thrombopoietin, which can stimulate the proliferation of hematopoietic stem cells.

The interleukin IL-6 refers to human interleukin-6, which can promote the proliferation of hematopoietic stem cells.

Wherein, the SFEM II medium refers to a serum-free basal medium for culturing hematopoietic stem cells from StemCell Company, which is suitable for culturing hematopoietic stem cells.

For example, the concentration of the growth factor Flt-3L can be 10 ng/ml, ng/ml, 30 ng/ml, 40 ng/ml, 50 ng/ml, 60 ng/ml, 70 ng/ml, 80 ng/ml, 90 ng/ml, 100 ng/ml, 110 ng/ml, etc.;
the concentration of the growth factor SCF can be 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 110 ng/ml, etc.;
the concentration of the growth factor TPO can be 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 110 ng/ml, etc.; and the concentration of the interleukin IL-6 can be 1 ng/ml, 5 ng/ml, 10 ng/ml, ng/ml, 30 ng/ml, 40 ng/ml, 50 ng/ml and the like.

In a preferred embodiment of the present application, the hematopoietic stem cell medium includes, for example, a serum-free basal medium, growth factor Flt-3L, growth factor SCF, growth factor TPO and interleukin IL-6 or the hematopoietic stem cell medium may include a serum-free basal medium, growth factor Flt-3L, growth factor SCF, and growth factor TPO.

The hematopoietic stem cell medium refers to a medium for culturing hematopoietic stem cells.

In a preferred embodiment of the present application, the concentration of the small molecule inhibitor of a PDGFR target in the culture medium composition is 0.1-100 μM, preferably 0.5-50 μM, more preferably 1-10 μM.

For example, the concentration of the small molecule inhibitor of a PDGFR target in the culture medium composition can be 0.1 μM, 0.5 μM, 1 μM, 2 μM, 3 μM, 4 μM, 5 μM, 6 μM, 7 μM, 8 μM, 9 μM, 10 μM, 15 μM, 20 μM, 30 μM, 40 μM, 50 μM, 60 μM, 70 μM, 80 μM, 90 μM, 100 μM, etc.

In a preferred embodiment of the present application, the HSCs are derived from bone marrow, mobilized peripheral blood, umbilical cord blood, cryopreserved and resuscitated HSCs or HSCs modified by gene editing.

The present application provides an HSCs infusion solution, wherein the cell number of CD34+ phenotype HSCs accounts for 40-85% of the total cells, preferably 60-85%, more preferably 75-80%.

For example, the cell number of CD34+ phenotype HSCs accounts for 40%, 45%, 50%, 55%, 60%, 65%, 70%, 75%, 80%, 85%, etc. of the total cells.

The whole cell refers to all progeny cells after the initial CD34+ cells are cultured.

In a preferred embodiment of the present application, the cell number of CD34+CD90+ phenotype HSCs accounts for 6-15% of the total cells, preferably 8-15%, more preferably 8-12%.

For example, the cell number of CD34+CD90+ phenotype HSCs accounts for 6%, 7%, 8%, 9%, 10%, 11%, 12%, 13%, 14%, 15%, etc. of the total cells.

In a preferred embodiment of the present application, wherein the cell number of CD34+CD90+CD45RA− phenotype HSCs accounts for 2-10% of the total cells, preferably 2-6%, more preferably 4-5%.

For example, the cell number of CD34+CD90+CD45RA− phenotype HSCs accounts for 2%, 3%, 4%, 5%, 6%, 7%, 8%, 9%, 10%, etc. of the total cells.

In a preferred embodiment of the present application, the cell number of CD34+CD45+CD90+CD45RA−CD38− phenotype HSCs accounts for 2-5% of the total cells, preferably 2.5-4%.

For example, the cell number of CD34+CD45+CD90+CD45RA−CD38-phenotype HSCs accounts for 2%, 3%, 4%, 5%, etc. of the total cells.

In a preferred embodiment of the present application, the HSCs are derived from bone marrow, mobilized peripheral blood, umbilical cord blood, cryopreserved and resuscitated HSCs or HSCs modified by gene editing.

The present application provides a method for replenishing blood cells to an individual in need, comprising infusing the above-mentioned HSCs infusion solution to the individual.

In some preferred embodiments of the present application, the colonization and differentiation of the HSCs in the individual are detected after the infusion of the HSCs infusion solution into the individual. In the method of the present application for replenishing blood cells to an individual in need, after the HSCs infusion solution is infused into the individual, the HSCs colonize and differentiate into blood cells in the individual. After the HSCs infusion solution is infused into the individual, whether the HSCs can be colonized and differentiated in the individual can be detected by a conventional method in the art for detecting the colonization and differentiation of HSCs. For example, after transplanting the mobilized peripheral blood hematopoietic stem cells, there will be two peaks in the increase of neutrophils. The first peak is about 11 days after transplantation, and the peripheral blood neutrophils reach 0.5×10 9/L, and then show a trend of decline. The second peak appears again 3 to 4 weeks after transplantation, and then it returns to normal. Therefore, whether HSCs are successfully colonized and differentiated after HSCs infusion can be judged by detecting the number of neutrophils in peripheral blood. The number of platelets in peripheral blood can also be detected to determine whether HSCs are successfully colonized and differentiated after HSCs infusion. For example, the average time after infusion is about 13 days to detect whether the peripheral blood platelets reach 50×10 9/L. After umbilical cord blood hematopoietic stem cell transplantation, it is judged by detecting whether the peripheral blood neutrophils reach 5×10⁹/L in an average of 22 to 24 days; or whether the peripheral blood platelets reach 5×10⁹ in an average of 48 to 54 days. Alternatively, the peripheral blood of the individual is continuously monitored, and the absolute neutrophil count for 3 consecutive days is ≥0.5×10⁹/L; or the platelet count is greater than 20×10⁹/L. In addition, there are also some indicators, such as sex chromosome transformation, blood type transformation, short tandem repeat (STR) turning to the donor type can also be used as a marker of successful implantation.

The present application provides a method for promoting the expansion of HSCs and maintaining the self-renewal capacity of HSCs, comprising in vitro culturing HSCs in a culture medium composition containing a small molecule inhibitor of a PDGFR target.

In the application, by in vitro culturing of HSCs in the culture medium composition containing the small molecule inhibitor of a PDGFR target, it can promote the expansion of HSCs and maintain the self-renewal ability of HSCs. In addition, after implanted in vivo, the expanded cells can differentiate into cells of different lineages.

In a preferred embodiment of the present application, the small molecule inhibitor of a PDGFR target is one or more selected from the group consisting of: AG1296, PDGFR inhibitor 1, Imatinib, PP121, Ponatinib, Axitinib, Trapidil and Erdafitinib; preferably AG1296.

In a preferred embodiment of the present application, wherein the culture medium composition comprises a hematopoietic stem cell medium, preferably, the hematopoietic stem cell medium comprises 1) a basal medium (preferably a serum-free basal medium) 2) a growth factor; and/or 3) a cytokine.

The growth factor or cytokine is one or more selected from the group consisting of: growth factor Flt-3L, growth factor SCF, growth factor TPO and interleukin IL-6;
preferably, the concentration of the growth factor Flt-3L is 10-110 ng/ml, preferably 50-100 ng/ml;
the concentration of the growth factor SCF is 10-110 ng/ml, preferably 50-100 ng/ml;
the concentration of the growth factor TPO is 10-110 ng/ml, preferably 50-100 ng/ml; and
the concentration of the interleukin IL-6 is 1-50 ng/ml, preferably 1-20 ng/ml.

In a preferred embodiment of the present application, wherein the concentration of the small molecule inhibitor of a PDGFR target in the culture medium composition is 0.1-100 μM, preferably 0.5-50 μM, more preferably 1-10 μM.

In a preferred embodiment of the present application, wherein the in vitro contact time is about 4-21 days, preferably about 6-15 days, more preferably about 6-10 days, most preferably about 6-8 days.

For example, the in vitro contact time can be about 4-21 days, about 4-20 days, about 4-19 days, about 4-18 days, about 5-21 days, about 5-20 days, about 5-19 days, about 5-18 days, about 6-18 days, about 6-17 days, about 6-16 days, about 6-15 days, about 6-14 days, about 6-13 days, about 6-12 days, about 6-11 days, about 6-10 days, about 6-9 days, about 6-8 days, etc.

In a preferred embodiment of the present application, wherein after the above-mentioned in vitro contact time, the cell number of CD34+ phenotype HSCs accounts for 40-85% of the total cells, preferably 60-85%, more preferably 75-80%.

In a preferred embodiment of the present application, wherein the cell number of CD34+CD90+ phenotype HSCs accounts for 6-15% of the total cells, preferably 8-15%, more preferably 8-12%.

In a preferred embodiment of the present application, wherein after the above-mentioned in vitro contact time, the cell number of CD34+CD90+CD45RA− phenotype HSCs accounts for 2-10% of the total cells, preferably 2-6%, more preferably 4-5%.

In a preferred embodiment of the present application, wherein after the above-mentioned in vitro contact time, the cell number of CD34+CD45+CD90+CD45RA−CD38− phenotype HSCs accounts for 2-5% of the total cells, preferably 2.5-4%.

In a preferred embodiment of the present application, the HSCs are derived from bone marrow, mobilized peripheral blood, umbilical cord blood, cryopreserved and resuscitated HSCs or HSCs modified by gene editing.

The present application uses a small molecule inhibitor of a PDGFR target to expand HSCs cells in vitro, and can expand cells from different sources, such as the above-mentioned HSCs cells derived from bone marrow, mobilized peripheral blood or umbilical cord blood, and cryopreserved and resuscitated HSCs or HSCs modified by gene editing.

The present application provides a cell population, wherein the proportion of CD34+ cells in the cell population is 40-85%.

The cell population refers to the in vitro cell product, which refers to a cell population obtained by contacting HSCs with a culture medium composition containing a small molecule inhibitor of a PDGFR target in vitro.

In a preferred embodiment of the present application, wherein the number of CD34+ cells accounts for 60-85% of the total cell population, preferably 75-80%.

In a preferred embodiment of the present application, wherein the cell population is obtained by culturing HSCs in vitro in a culture medium composition containing a small molecule inhibitor of a PDGFR target.

The cell population is capable of maintaining sternness, and after implantation in vivo, it can differentiate into cells of different lineages for the treatment of different diseases.

In a preferred embodiment of the present application, wherein the small molecule inhibitor of a PDGFR target is one or more selected from the group consisting of: AG1296, PDGFR inhibitor 1, Imatinib, PP121, Ponatinib, Axitinib, Trapidil and Erdafitinib; preferably AG1296.

In a preferred embodiment of the present application, wherein the culture medium composition comprises a hematopoietic stem cell medium, preferably, the hematopoietic stem cell medium comprises: 1) a basal medium (preferably a serum-free basal medium); 2) a growth factor; and/or 3) a cytokine;
the growth factor or cytokine is one or more selected from the group consisting of: growth factor Flt-3L, growth factor SCF, growth factor TPO and interleukin IL-6;
preferably, the concentration of the growth factor Flt-3L is 10-110 ng/ml, preferably 50-100 ng/ml;
the concentration of the growth factor SCF is 10-110 ng/ml, preferably 50-100 ng/ml;
the concentration of the growth factor TPO is 10-110 ng/ml, preferably 50-100 ng/ml; and
the concentration of the interleukin IL-6 is 1-50 ng/ml, preferably 1-20 ng/ml.

In a preferred embodiment of the present application, wherein the concentration of the small molecule inhibitor of a PDGFR target in the culture medium composition is 0.1-100 μM, preferably 0.5-50 μM, more preferably 1-10 jam.

In a preferred embodiment of the present application, wherein the HSCs are derived from bone marrow, mobilized peripheral blood, umbilical cord blood, cryopreserved and resuscitated HSCs or HSCs modified by gene editing.

In a preferred embodiment of the present application, the cell population differentiates into blood cells of different lineages after implantation in vivo. For example, it can differentiate into B cells, T cells, NK cells, dendritic cells, granulocytes, macrophages, megakaryocytes or erythrocytes.

The present application provides a method for preventing or treating a disease of an individual, comprising infusing the above-mentioned HSCs infusion solution or the above-mentioned cell population to the individual.

The present application provides a use of a small molecule inhibitor of a PDGFR target in promoting the expansion of HSCs and maintaining the self-renewal ability of HSCs, preferably, the small molecule inhibitor of a PDGFR target is one or more selected from the group consisting of: AG1296, PDGFR inhibitor 1, Imatinib, PP121, Ponatinib, Axitinib, Trapidil, and Erdafitinib; preferably AG1296.

In a preferred embodiment of the present application, wherein the HSCs are derived from bone marrow, mobilized peripheral blood, umbilical cord blood, cryopreserved and resuscitated HSCs or HSCs modified by gene editing.

The present application provides a use of the above-mentioned HSCs infusion solution or the above-mentioned cell population in the preparation of a medicament for preventing or treating diseases.

Preferably, the disease is a disease requiring replenishment of blood cells.

In a preferred embodiment of the present application, wherein when the blood cells are red blood cells, anemia and the like can be treated;

In a preferred embodiment of the present application, wherein when the blood cells are white blood cells, leukopenia, agranulocytosis, eosinophilia, acute leukemia, chronic leukemia, myelodysplastic syndrome, malignant lymphoma (Hodgkin's lymphoma, non-Hodgkin's lymphoma), infectious mononucleosis, malignant histiocytosis, multiple myeloma, and the like can be treated;

In a preferred embodiment of the present application, wherein when the blood cells are platelets, aplastic anemia, acute leukemia, acute radiation sickness, and the like can be treated;

The PDGFR inhibitor of the present application can significantly expand HSCs during in vitro culture, while maintaining a high proportion of the self-renewal capacity of HSCs. In addition, the PDGFR inhibitor can expand cells from different sources in vitro, and after the expanded cells are implanted in vivo, they can differentiate into cells of different lineages, and can widely treat a series of blood system diseases.

In this application, the term LT-HSCs is the abbreviation of Long Term Hemopoietic Stem Cells, which refers to a class of stem cells with high differentiation potential that are in a quiescent state and capable of self-renewal, and can support the reconstruction of long-term hematopoietic system, for example reconstruction of recipient hematopoietic system in secondary transplantation.

The self-renewal ability and differentiation potential of hematopoietic stem cells (HSCs) can be called the “sternness” of hematopoietic stem cells. The present application found that among the CD34+ hematopoietic stem cells, LT-HSCs are a group of cells in hematopoietic stem cells with the best self-renewal ability and differentiation potential, and can support the reconstruction of the long-term hematopoietic system.

### EXAMPLES

The present application generally and/or specifically describes the materials used in the test and test methods. In the following examples, unless otherwise specified, % in the chemical materials used means wt %, that is, weight percentage. The reagents or machines used without the manufacturer's indication are all conventional reagents and products that can be obtained from the market.

### Example 1 CD34+HSCS Sorted from Umbilical Cord Blood/Mobilized Peripheral Blood for Subsequent Small Molecule Screening

Preparation of reagents H-lyse Buffer (1×) solution and Wash Buffer (1×) solution: 5 ml of H-lyse Buffer 10× stock solution (R&D, Cat. No.: WL1000) was taken, then adding 45 ml of deionized water (EdiGene, 0.22 μm filter membrane) to mix well to prepare an H-lyse Buffer (1×) solution;

5 ml of Wash Buffer 10× stock solution (R&D, Cat. No.: WL1000) was taken, then adding 45 ml of deionized water to mix well to prepare a Wash Buffer (1×) solution.

Physiological saline was added to 10 ml of umbilical cord blood/mobilized peripheral blood (EdiGene) to a final volume of 30 ml. Human lymphocyte separation solution (Dakewe, Cat. No.: DKW-KLSH-0100) was added to the diluted blood, and then centrifuging at 400 g for 30 min (seting acceleration speed 3, deceleration speed 0), sucking the buffy coat to centrifuge at 500 g for 10 min. The cell pellet were collected into a 50 ml centrifuge tube, adding 10 ml of H-lyse Buffer (1×), lysing the red blood cells at room temperature for 10 minutes. Then 10 ml of Wash Buffer (1×) was added to stop the lysis reaction, and adding physiological saline to make a final volume of 50 ml. The above 50 ml centrifuge tube was transferred to a high-speed centrifuge, centrifuging at 500 g for 10 min, discarding the supernatant, resuspending the cells with 50 ml of physiological saline (1% HSA), mixing well, and taking 20 μL of the cell suspension to a cell counter (Nexcelom, model: Cellometer K2) for counting; transferring the centrifuge tube to a high-speed centrifuge, and centrifuging at 500 g for 10 min. The supernatant was discarded and added a corresponding volume of magnetic beads (100 μL FcR/1*10\hat{ }8 cells and 100 μL CD34 MicroBeads/1*10\hat{ }8 cells) according to the counting result. The operation was as follows: First, FcR Blocking Reagent (Miltenyi Biotec, Cat. No.: 130-100-453, the amount of reagent is determined according to the cell count results) was added to resuspend the cells, then adding pre-mixed CD34 MicroBeads (CD34 MicroBead Kit UltraPure, human. MiltenyiBiotec, Cat. No.: 130-100-453), mixing well, and incubating in the refrigerator at 4° C. for 30 min Physiological saline (1% HSA) was assed to the centrifuge tube to a final volume of 50 ml, then transferring to a high-speed centrifuge, and centrifuging at 500 g for 10 min. A magnetic separator (MiltenyiBiotec, model: 130-042-102) and a magnetic stand (MiltenyiBiotec, model: 130-042-303) were provided, adjusting the magnetic separator to a suitable height, and putting it into the MS Column (MiltenyiBiotec, Cat. No.: 130)-042-201) or LS Column (MiltenyiBiotec, Item No.: 130-042-401) (the type of column should be selected according to the amount of cells, the details are referred to the relevant product instructions), and placing a 15 ml centrifuge tube (Corning, Item No.: 430791) below to collect non-target cell suspension, and rinsing the MS Column or LS Column with 1 ml (MS column) or 3 ml (LS column) of physiological saline (1% HSA). After centrifugation in the centrifuge tube in the above-mentioned high-speed centrifuge (Thermo, model: ST40), the supernatant was discarded, and the cells were resuspended in 1 ml (MS column) or 3 ml (LS column) of physiological saline (1% HSA), adding the cell suspension to each sorting column (the amount of the sorting column was determined according to the number of umbilical cord blood/mobilized peripheral blood and the amount of cells). The centrifuge tubes were washed with 1 ml (MS column) or 3 ml (LS column) of physiological saline (1% HSA), and the washing solution was added to the column.

The MS Column or LS Column was washed with 1 ml (MS column) or 3 ml (LS column) of physiological saline (1% HSA), repeating 3 times. The sorting column was transferred to the top of a new 15 ml centrifuge tube, adding 2 ml (MS column) or 3 ml (LS column) of physiological saline (1% HSA) to elute the target cells, and then adding 1 ml (MS column) or 2 ml (LS column) to elute the target cells again with physiological saline (1% HSA). 20 μL of the cell suspension was taken into a cell counter (Nexcelom, model: Cellometer K2) for counting, and the remaining cell suspension was centrifuged at 400 g for 5 min. The supernatant was incompletely discarded to keep 1 ml of the supernatant, and resuspending the cells. A new MS Column was taken, adding 1 ml of physiological saline (1% HSA) to rinse, transferring the cell suspension of the resuspended cells to the MS Column, repeating the above washing and elution steps to obtain 3 ml of the target cell suspension. 20 μL of cell suspension was taken to count in a cell counter (Nexcelom, model: Cellometer K2), calculating the total number of cells according to cell density and cell suspension volume; the remaining cell suspension was centrifuged at 400 g for 5 min, and discarding the supernatant for later use.

### Example 2 Concentration Test and Screening of Small Molecule Inhibitor

According to the solubility and required solvent indicated in the instructions of the small molecule inhibitor (see Table 1 for the Cat. number of the small molecule inhibitor), the preparation of the small molecule inhibitor stock solution was carried out. Next, the hematopoietic stem cell medium was prepared: SFEM II medium (stem cell, Cat. number: 09655)+50 ng/ml growth factor Flt-3L (PeProtech, Cat. number: 300-100UG)+50 ng/ml growth factor SCF (PeProtech, Cat. number): 300-07-100UG)+50 ng/ml growth factor TPO (PeProtech, Cat. number: 300-18-100UG)+10 ng/ml interleukin IL-6 (PeProtech, Cat. number: 200-06-20UG)+1% bispecific antibody (HyClone, Cat. No.: sv30010). According to the set concentration gradient of the small molecule inhibitor, the stock solution and the basal medium were used to prepare the medium containing different concentrations of the small molecule inhibitor.

First, the prepared medium was added to a 24-well plate (Corning, Cat. number: 3473), 950 μl per well, and placing it in a carbon dioxide incubator (Thermo, model: 3111) to preheat; resuspending the spare umbilical cord Blood-derived HSCs of Example 1 with SFEM 11+50 ng/ml Flt-3L+50 ng/ml SCF+50 ng/ml TPO+10 ng/ml IL-6+1% bispecific antibody, calculating the medium volume to be added according to 50 μl cell suspension per well and 2*10\hat{ }5/m1 cell density per well. For example, when the final volume of the cell culture solution per well was 1 ml, the total number of cells per well was 2*10\hat{ }5 cells calculated according to the cell density per well, and the density of the 541 of cell suspension supplemented to each well is 4*10\hat{ }6/ml. The density of the spare HSCs of Example 1 was adjusted to the calculated density of the cell suspension, and adding it; taking out the preheated medium from the incubator, adding 50 μl of the cell suspension to each well, and after mixing, using the microscope (OLYMPUS, model: CKX53) to observe the cells state, and then putting it into an incubator for culturing.

TABLE 1
Small Molecule Inhibitors

| Name of Small | | | |
|---|---|---|---|
| Molecule Inhibitor | Action pathway/Target | Cat. No. | Brand |
| Scutellarin | NOS | 73577 | sigma |
| NG-Methyl-L-Arginine | NOS | M7033 | sigma |
| acetate salt | | | |
| 1-Methylnicotinamide | NOS | SML0704 | sigma |
| chloride | | | |
| RG7834 | HBV Inhibitor | HY-117650A | MCE |
| PFI-2 | lysine methyltransferase | S7294 | Selleck |
| | SETD7 inhibitor | | |
| Salirasib | prenylated protein | S8460 | Selleck |
| | methyltransferase | | |
| | (PPMTase) inhibitor | | |
| UNC0379 | N-lysine methyltransferase | S7570 | Selleck |
| | SETD8 inhibitor | | |
| CCN3 | Ligand of Notch signaling | 1640-NV-050 | R&D |
| | pathway | | |
| BVT948 | methyltransferase SETD8 | #2176/10 | R&D |
| | inhibitor | | |
| Sunitinib | FLT3, c-Kit, PDGFRβ, | S7781 | Selleck |
| | VEGFR2 | | |
| SAG | Smoothened | S7779 | Selleck |
| AG1296 | PDGFR | S8024 | Selleck |
| Tamibarotene | Retinoic acid receptor | S4260 | Selleck |
| | (RAR) agonist | | |
| AM580 | Retinoic acid receptor | S2933 | Selleck |
| | agonist | | |
| Tretinoin | Retinoic acid receptor | S1653 | Selleck |
| Anti-hEndomucin | Endomucin | AF7206-SP | R&D |
| PDGFR inhibitor 1 | PDGFR | S8721 | Selleck |
| Imatinib (STI571) | PDGFR | S2475 | Selleck |
| PP121 | PDGFR | S2622 | Selleck |
| Cynarin | GSH/ROS | S3301 | Selleck |
| Bezafibrate | PPAR agonist | S4159 | Selleck |
| Pioglitazone HCl | cytochrome P450 | S2046 | Selleck |
| | Inhibitor | | |
| Diphenyleneiodonium | Inhibitor of NADPH | S8639 | Selleck |
| chloride (DPI) | oxidase | | |
| CAY10602 | SIRT1 agonist | S5918 | Selleck |
| Salermide | Reverse amide | S8460 | Selleck |
| Flubendazole | Flubendazole is an | S1837 | Selleck |
| | autophagy inducer by | | |
| | targeting Atg4B, used to | | |
| | treat internal parasite and | | |
| | worm infection. | | |
| Olaparib | Autophagy and mitophagy | S1060 | Selleck |
| | agonist | | |
| Torkinib | mTORC1/C2 Inhibitor | S2218 | Selleck |
| P62-mediated | Mitophagy regulator | HY-115576 | MCE |
| mitophagy inducer | | | |

### Example 3 Flow Cytometry Detection for Hscs Stemness and CD34+ Maintenance

The antibodies used in this example and their sources were shown in Table 2.

TABLE 2
Antibodies

| Name of antibody | Manufacturer | Cat. No. |
|---|---|---|
| APC/Cy7 anti-human CD45 | Biolegend | 304014 |
| APC anti-human CD38 | Biolegend | 356606 |
| Brilliant Violet 510&#8482; anti-human CD34 | Biolegend | 343528 |
| PE anti-human CD90 (Thy1) | Biolegend | 328110 |
| FITC anti-human CD45RA | Biolegend | 304106 |
| APC Mouse IgG2a, κ Isotype Ctrl | Biolegend | 400220 |
| APC/Cyanine7 Mouse IgG1, κ Isotype Ctrl | Biolegend | 400128 |
| PE Mouse IgG2a, κ Isotype Ctrl | Biolegend | 400212 |
| FITC Mouse IgG2b, κ Isotype Ctrl | Biolegend | 402208 |
| Brilliant Violet 510&#8482; Mouse | Biolegend | 400268 |
| IgG2a, κ Isotype Ctrl | | |

20 μL of cells cultured for 6-8 days (D6-D8) in the above Example 2 was sampled for counting, taking out a suspension of 2*10\hat{ }5 cells into a 1.5 ml centrifuge tube according to the counting result; centrifuging at 400 g for 5 min, discarding the supernatant. 100 μL of PBS (phosphate-buffered saline solution, HyClone, Cat. number: SH30256.01) containing 1% HSA (human serum albumin, Guangdong Shuanglin, Cat. number: S10970069) was taken, resuspending the cells, and vortexing to mix well for later use. Then, the cell sample in the control was collected. The number of cells and the collection method were the same as those of the sample cells to be tested. The controls were setting as the NC group and the ISO group respectively, selecting the cells from any sample or mixed cells of the samples to be tested in this batch of experiments, depending on the number of cells. In the same batch of experiments, each control group did not have repeated detection. The group settings were shown in Table 3.

TABLE 3

| | | | Amount of |
|---|---|---|---|
| | Cell | | the |
| Group | number | Name of the Antibody added | antibody |
| NC | 2 × 10^{∧}5 | — | — |
| ISO | 2 × 10^{∧}5 | APC Mouse IgG2a, κ Isotype Ctrl | 2 μL |
| | | APC/Cyanine7 Mouse IgG1, κ Isotype Ctrl | 2 μL |
| | | PE Mouse IgG2a, κ Isotype Ctrl | 2 μL |
| | | FITC Mouse IgG2b, κ Isotype Ctrl | 2 μL |
| | | Brilliant Violet 510&#8482; Mouse | 2 μL |
| | | IgG2a, κ Isotype Ctrl | |
| FMO38 | 2 × 10^{∧}5 | APC/Cy7 anti-human CD45 | 2 μL |
| | | Brilliant Violet 510&#8482; | 2 μL |
| | | anti-human CD34 | |
| | | PE anti-human CD90 (Thy1) | 2 μL |
| | | FITC anti-human CD45RA | 2 μL |
| FMO90 | 2 × 10^{∧}5 | APC/Cy7 anti-human CD45 | 2 μL |
| | | APC anti-human CD38 | 2 μL |
| | | Brilliant Violet 510&#8482; | 2 μL |
| | | anti-human CD34 | |
| | | FITC anti-human CD45RA | 2 μL |
| sample | 2 × 10^{∧}5 | APC/Cy7 anti-human CD45 | 2 μL |
| | | APC anti-human CD38 | 2 μL |
| | | Brilliant Violet 510&#8482; | 2 μL |
| | | anti-human CD34 | |
| | | PE anti-human CD90 (Thy1) | 2 μL |
| | | FITC anti-human CD45RA | 2 μL |

According to Table 3 above, the antibodies were added to the cell suspensions of the above-mentioned cell samples to be tested and control cell samples according to groups; vortexing to mix and incubating at room temperature for 15 min in the dark. After 15-min incubation, 1 ml of PBS containing 1% HSA was added to each test sample, mixing well, centrifuging at 400 g for 5 min at room temperature. After centrifugation, the supernatant was discarded, and the cells were resuspended in 100 μL of PBS containing 1% HSA for each test sample. Samples were stored at room temperature away from light before testing. Flow cytometry was used for detection.

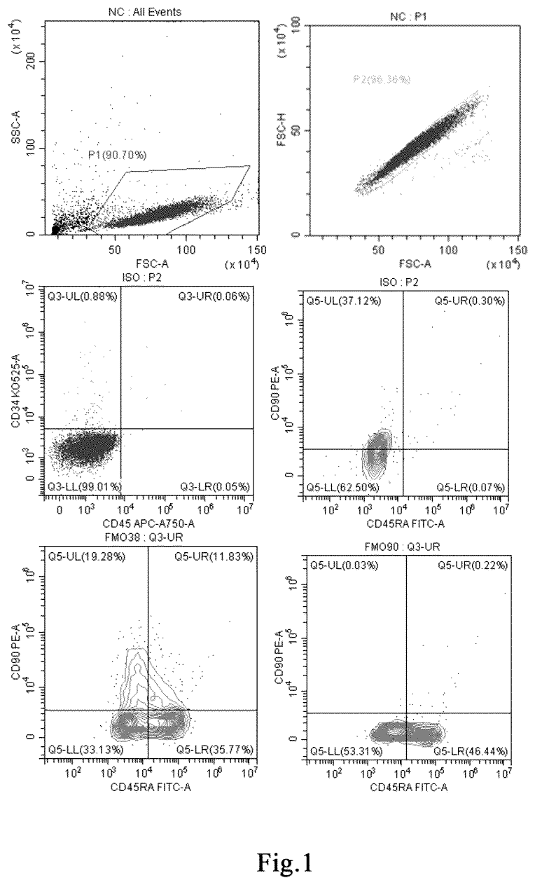

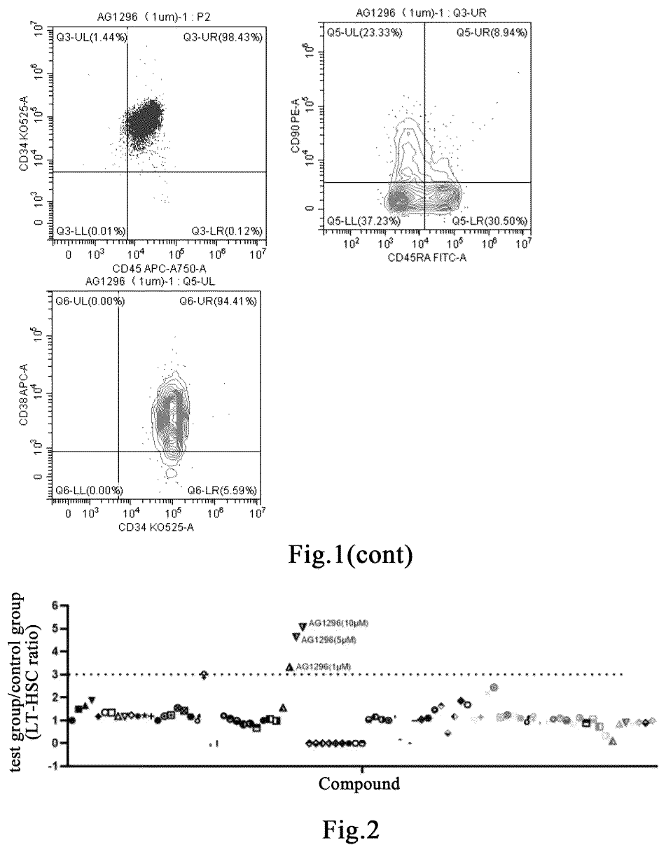

The test results were analyzed as follows: 1) The target cell population was CD34+CD45+CD45RA−CD90+CD38− cell population; 2) The determination of the logic gate and gate position was shown in FIG. 1: firstly delineating the cell population, P1 gate; removing adherent cells from the cell population of the P1 gate, as P2 gate; delineating the cell population from the P2 gate with NC or ISO for CD34, CD45, CD45RA negative cell population as the Q3-LL gate (CD34-CD45−), and Q5-UL+Q5-LL gate (CD45RA−); using FMO90 to delineate the CD90-negative cell population as Q5-LL+Q5-LR gate; using FMO38 to delineate the CD38-negative cell population as Q6-LR gate; using the gates delineated by NC, ISO, and FMO, it is determined that the cells delineated by the Q3-UR-Q5-UL-Q6-LR gate are CD34+CD45+CD45RA−CD90+CD38-target cells.

### Example 4 Preliminary Screening of Small Molecule Inhibitors

On the umbilical cord blood-derived CD34+ cells sorted in Example 1, the optimal concentrations of small molecule inhibitors and the ability to maintain the stemness of HSCs were screened in the same way as in Example 2. After 6-8 days of small molecule induction, the expression of LT-HSCs cell surface markers (CD34+CD45+CD90+CD45RA−CD38−) was detected by flow cytometry using the same method as in Example 3.

A total of 29 small molecules were screened in this example (see Table 1), and 3 concentrations of each inhibitor were tested.

The results in FIG. 2 show that the inhibitors represented by the dots above the dashed line can maintain the sternness of HSCs well, which is more than 3 times that of Mock in the negative control group. The three different triangles above the dashed line represent different concentrations of AG1296, and the concentrations used are indicated.

In conclusion: in this example, a small molecule that can maintain the sternness of LT-HSCs was screened out, which is AG1296, an inhibitor targeting PDGFR.

### Example 5: Screening of the Optimal Concentration of the Screened-Out PDGFR Inhibitor AG1296

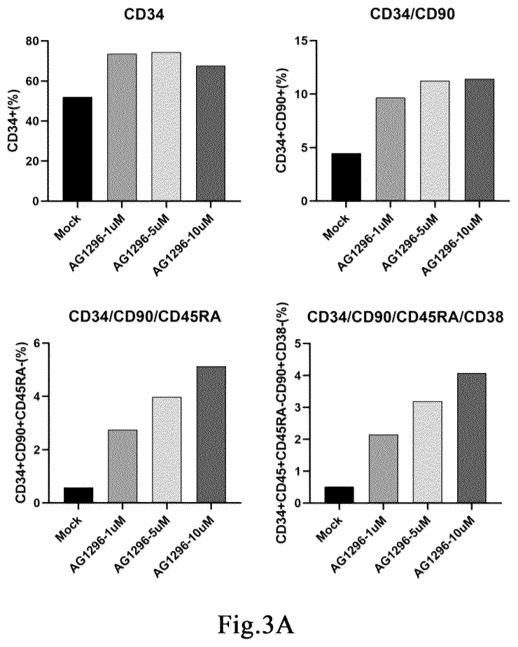

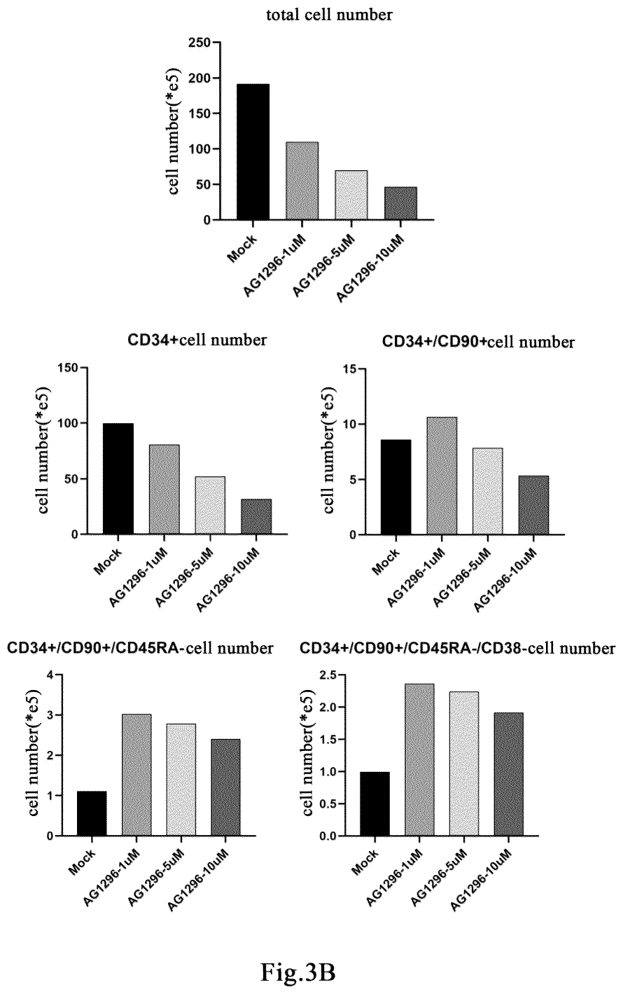

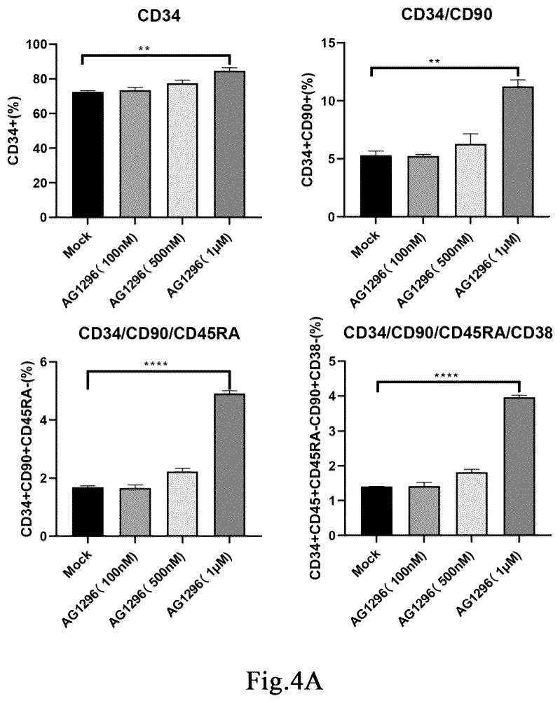

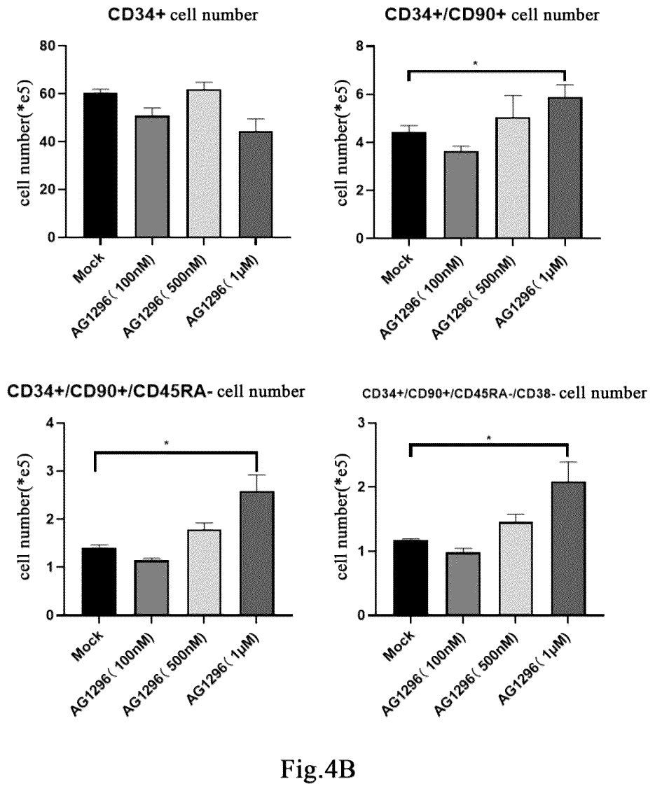

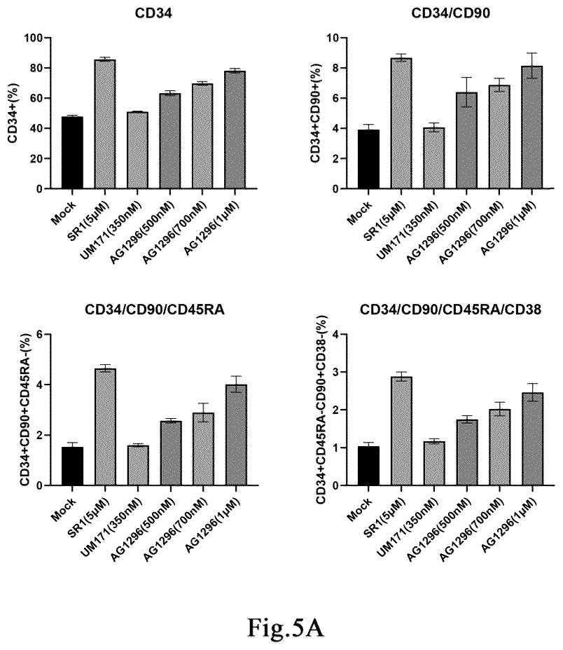

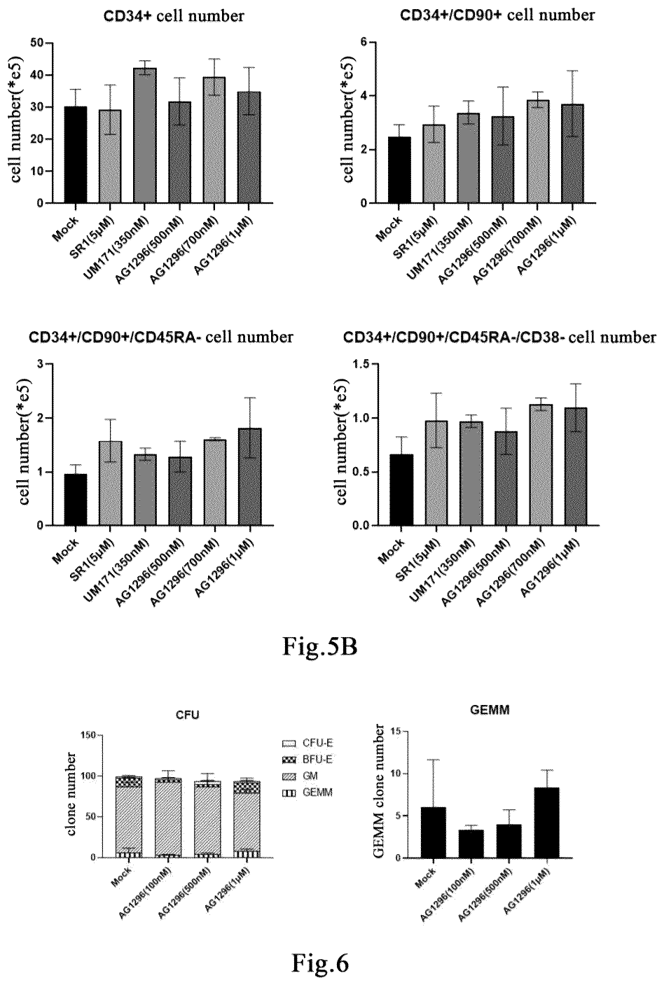

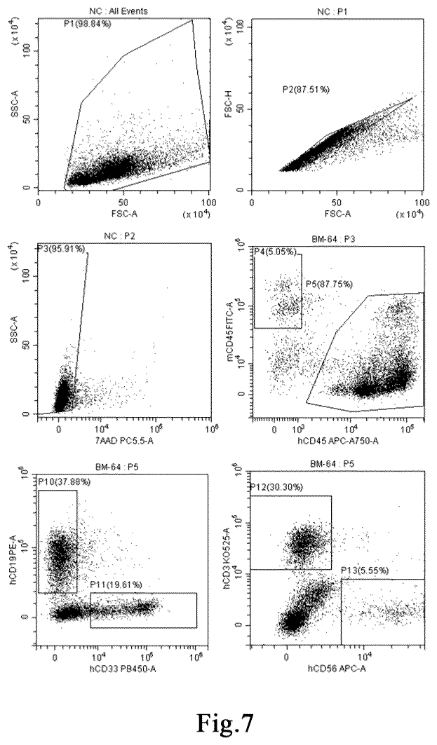

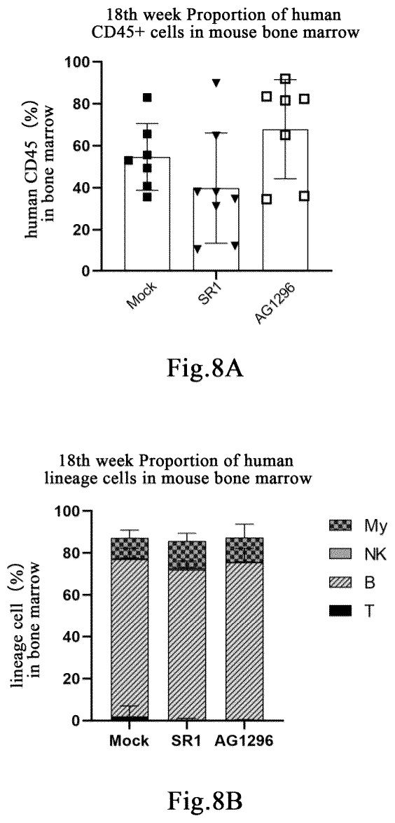

On the umbilical cord blood-derived CD34+ cells sorted in Example 1, the optimal concentration of the screened-out inhibitor AG1296 was screened in the same way as in Example 2. After 6 days of induction with different concentrations of the small molecule inhibitor AG1296, the expression of LT-HSCs cell surface markers (CD34+CD45+CD90+CD45RA−CD38−) was detected by flow cytometry using the same method as in Example 3. 20 μL of cell suspension was taken to count in a cell counter (Nexcelom, model: Cellometer K2) on the 6th day of culture, calculating the absolute number of the final CD34+ cells and LT-HSCs on the 6th day (absolute number of cells=total number of cells*sternness ratio). The results were shown in FIGS. 3A-3B and FIGS. 4A-4B, respectively.

The results in FIG. 3A showed that AG1296 was superior to the Mock group at 1 μM, 5 μM and 10 μM concentrations in maintaining the CD3+, CD34+CD90+, CD34+CD90+CD45RA− cell ratios.

The results in FIG. 3B showed that in the total cell number, AG1296 (1 μM) had higher cell numbers than that at concentrations of 5 μM and 10 μM, and in maintaining absolute numbers of CD34+ cells, AG1296 at 1 μM, 5 μM and 10 μM concentrations were lower than that in the mock group. It was proved that AG1296 slightly inhibited cell proliferation at the concentrations of 1 μM, 5 μM and 10 μM, but in terms of maintaining the absolute number of CD34+CD90+CD45RA− cells, AG1296 (1 μM, 5 μM and 10 μM) was significantly better than the Mock group. In terms of absolute number of proliferating LT-HSCs, AG1296 (1μM, 5μM and 10μM) had better effect.

The results in FIG. 4A showed that in maintaining the ratio of CD34+, CD34+CD90+, and CD34+CD90+CD45RA− cells, AG1296 at the concentration of 1 μM was better than Mock group and at the concentrations of 100 nM and 500 nM, with significant difference. In terms of increasing the proportion of LT-HSC, AG1296 (1 μM) was about 3 times that of Mock group and AG1296 (100 nM) group, and about 2 times that of AG1296 (500 nM), which significantly increased the proportion of LT-HSC.

The results in FIG. 4B showed that in maintaining the absolute number of CD34+ cells, AG1296 (1 μM) was not significantly better than other groups, proving that AG1296 slightly inhibited cell proliferation at a concentration of 1 μM, but in terms of maintaining the absolute number of CD34+CD90+, and CD34+CD90+CD45RA− cells, AG1296 (1 μM) was significantly better than other groups. In terms of the absolute number of proliferating LT-HSCs, the effect of AG1296 (1 μM) was 1-2 times that of other groups.

In conclusion, AG1296 at concentrations of 1 μM, 5 μM and 10 μM was better in maintaining the sternness and absolute cell number of LT-HSCs on umbilical cord blood-derived HSCs.

### Example 6: Comparison of the Screened-Out PDGFR Inhibitor AG1296 and Literature-Reported Inhibitors UM171 and SR1

On the mobilized peripheral blood-derived CD34+ cells sorted in Example 1, the screened-out inhibitor AG1296 and literature-reported (Fares I, et al. Science. 2014; Boitano A E, et al. Science. 2010) inhibitors UM171 and SR1 were compared in the same way as Example 2. Eight days after the induction of the small molecule inhibitor, the expression of LT-HSCs cell surface markers (CD34+CD45+CD90+CD45RA−CD38−) was detected by flow cytometry using the same method as in Example 3. After 8 days of culture, 20 μL of cell suspension was taken into a cell counter (Nexcelom, model: Cellometer K2) and counted, and the absolute numbers of the final CD34+ cells and LT-HSCs on day 6 were calculated (absolute number of cells=total number of cells*stemness ratio). The results were shown in FIG. 5.

The results in FIG. 5A showed that AG1296 (1 μM) was significantly more effective than Mock group, UM171 group, AG1296 (500 nM) group and AG1296 (700 nM) group in maintaining the proportion of CD34+, CD34+CD90+, and CD34+CD90+CD45RA− cells, but not as good as the SR1 group. In terms of increasing the proportion of LT-HSCs, AG1296 (1 μM) was about 2 times that of Mock group and UM171 group, and about 1 time that of AG1296 (500 nM) group and AG1296 (700 nM) group, significantly increasing the proportion of LT-HSCs, but the effect was not as good as SR1 group.

The results in FIG. 5B showed that in maintaining the absolute number of CD34+ cells, AG1296 (1 μM) was not significantly better than other groups, proving that AG1296 could inhibit cell proliferation at a concentration of 1 μM, and the UM171 group had the best effect. But in maintaining the absolute number of CD34+CD90+, and CD34+CD90+CD45RA− cells, AG1296 (1 μM) showed superiority.

### Example 7: CD34+ Hematopoietic Stem Cell Colony Formation and Culture

In this example, the colony-forming unit (CFU) was used to detect the in vitro function of cord blood-derived hematopoietic stem cells induced by a small molecule inhibitor for qualitative and quantitative detection to verify their in vitro differentiation potential.

First, 100 mL of medium MethoCult™ H4034 Optimum (Stem Cell, Cat. No.: 04034) were aliquoted, then thawing at 2-8° C. overnight; shaking vigorously for 1-2 minutes and letting it stand for 10 minutes until the bubbles rised to the liquid level. After tightly fitting the 50 mL syringe needle to the 5 mL disposable syringe, the medium is aspirated to 1 mL, pushing out the syringe completely to exhaust the gas in the syringe, and re-absorbing 3 mL into each 15 mL centrifuge tube (Corning, Cat. No. 430791); storing at 2-8° C. for 1 month, and at −20° C. for long-term storage. Do not freeze and thaw repeatedly.

3 mL of medium Meth® Cult′ H4034 Optimum was prepared and thawed overnight at room temperature (15-25° C.) or 2-8° C.

Cell seeding was carried out: the cell suspension after 7 days of expansion and culturing after induction by small molecule inhibitors (cord blood-derived CD34+ hematopoietic stem cells induced by small molecule inhibitors) was taken for cell counting, then aspirating cells suspension with 100 times the seeding density according to the counting results (for example, if the seeding density was 100 cells/well/3 ml, 10000 cells should be collected), adding them to 1 ml of 2% FBS (Gibco, Cat. No. 16000-044)-IMDM (Gibco, Cat. No.: 12440-053) medium, mixing for use. After mixing the above cells well, 50 μL of the cell suspension was aspirated into 0.5 mL of IMDM (2% FBS) to resuspend the cells (equivalent to 10-fold dilution of the cell suspension). After mixing well, 100 μL of the cell suspension (100 cells) was taken out to add into 3 mL of MethoCult™ H4034 Optimum; vortexing for at least 4 s and letting stand for 10 min, until the bubbles rised to the liquid level. 3 cc Syringes (Stem cell, Item No.: 28240) was used in conjunction with Blunt-End Needles 16 Gauge (Stemcell, Item No.: 28110), aspirating the obtained cell suspension to 1 mL, pushing out the syringe completely to exhaust the gas in the syringe, and re-absorbing all the obtained cell suspension. 3 mL was injected into one well of SmsrtDish™-6 (stem cell, Cat. number: 27370, 6-well plate), and tilting the 6-well plate slowly so that the cell suspension evenly covered the bottom of the well. After seeding all cells as above, 3 ml of sterile PBS was added to gaps between wells of the 6-well plate to prevent the medium from drying up. The 6-well plate was covered with a lid and placing it in a carbon dioxide incubator (Thermo, model: 3111) to culture for 14 days at 37° C., 5% CO₂, and 95% relative humidity.

Colonies were observed on the 7th and 14th days of culture, and colonies were counted with a STEMgrid™-6 counting grid (stem cell, Cat. number: 27000) after 14 days of culture. The criteria for determining colonies were as follows (different types of colonies could reflect the ability of HSCs to form colonies and maintain the stemness):

CFU-GEMM (CFU-G, CFU-E, CFU-MM): granulocyte-erythrocyte-macrophage-megakaryocyte colony forming unit. A colony contains erythrocytes and 20 or more non-erythrocytes (granulocytes, macrophages, and/or megakaryocytes), usually with erythrocytes in the center of the colony, surrounded by non-erythrocytes, and non-erythrocytes could also be concentrated on one side of the erythrocytes. Colonies of CFU-GEMM were generally larger than those of CFU-GM or BFU-E. They were rare in most cell samples (usually 10% of total colonies).

CFU-GM: Colonies contained more than 20 granulocytes (CFU-G) and/or macrophages (CFU-M). Without appearing red or brown, individual cells within colonies were often distinguishable, especially at the colony edges, and large colonies may have one or more dense dark nuclei. Erythropoietin (EPO) was not required for colony growth and differentiation.

BFU-E: Burst erythrocyte colony-forming unit, forming colonies consisted of single or multiple cell clusters, each colony containing >200 mature erythrocytes. When cells were hemoglobinated, they appeared red or brown, making it difficult to distinguish individual cells within each cluster. BFU-E were more immature progenitor cells that require erythropoietin (EPO) and other cytokines, especially interleukin 3 (IL-3) and stem cytokine (SCF), for their growth to promote optimal growth of their colonies.

CFU-E: erythrocyte colony-forming unit, which could form 1-2 cell clusters containing 8-200 red blood cells. When the cells were hemoglobinated, they appeared red or brown, and it was difficult to distinguish individual cells within the colony. CFU-E were progenitors of the mature erythroid lineage and required erythropoietin (EPO) to promote their differentiation.

### Example 8: Comparison of the Effects of the Screened-Out PDGFR Inhibitor AG1296 on the In Vitro Clonogenic Ability of HSCS

The comparison of the effects of different concentrations of the screened-out PDGFR inhibitor AG1296 on the in vitro clonogenic ability of HSCS was performed on the umbilical cord blood-derived CD34+ cells sorted in Example 1. Cells were treated with different concentrations of AG1296, and after 8 days, in vitro clone (CFU) formation detection was carried out in the same way as in Example 7, the number of clones was counted 14 days after seeding cells, and CFU-GEMM was analyzed. The results were shown in FIG. 6, among which, BFU-E, CFU-E, CFU-GM, CFU-GEMM represented clones of different lineages of blood system such as erythroid, myeloid, and lymphoid.

The results in FIG. 6 showed that, in terms of the total number of clones, there was little difference between the groups. In terms of the number of GEMM clones differentiated from LT-HSCs, AG1296 (1 μM) was significantly better than the other groups. GEMM clones represented the ability of hematopoietic stem cells to differentiate into cells of other lineages. The greater the number of GEMM clones, the stronger the self-renewal and transplantation reconstruction ability of hematopoietic stem cells. In conclusion, AG1296 could well maintain the self-renewal capacity and the absolute cell number of LT-HSCs during the in vitro expansion of HSCs.

### Example 9: Comparison of the Effects of the Screened-Out PDGFR Inhibitor AG1296 and the Inhibitor SR1 Reported in the Literature on Hematopoietic Stem Cell Transplantation In Vivo

On the umbilical cord blood-derived CD34+ cells sorted in Example 1, the effects of the screened small molecule inhibitor AG1296 and the inhibitor SR1 reported in the literature on the in vivo hematopoietic system reconstitution ability was compared. The concentrations and groups of small molecule inhibitors used in this example were shown in Table 4.

TABLE 4
Small molecule inhibitor concentrations

| | Concentration of small |
|---|---|
| Group | molecule inhibitor |
| Mock | NA |
| SR1 | 5 μM |
| AG1296 | 1 μM |

Preparation of cell medium: SFEM II+100 ng/ml Flt-3L+100 ng/ml SCF+100 ng/ml TPO+20 ng/ml IL-6+1% bispecific antibody. Cat. numbers of the medium, growth factor, bispecific antibody, etc. were as described in Example 2. According to the groups set in Table 4, different small molecule inhibitors were added.

The prepared cell medium was added to a 24-well plate, 950 μl per well, and placing it in a carbon dioxide incubator to preheat; using SFEMII+100 ng/ml Flt-3L+100 ng/ml SCF+100 ng/ml TPO+20 ng/ml IL-6+1% bispecific antibody to resuspend the spare umbilical cord blood-derived HSCs of Example 1. The medium volume required to resuspend the cells was calculated according to 50 μl of cell suspension was added to each well, and 0.28*10\hat{ }5/ml cell density per well was; taking out the preheated medium from the incubator, adding 50 μl of resuspended cell suspension to each well, mixing well, observing the cell state under a microscope, and then putting them into the incubator for culture. The initial number of culture cells transplanted in each mouse was 0.28*10\hat{ }5/mouse, that was, the cells expanded in each well of a 24-well plate could be transplanted into one mouse. Counting every other day during the cell culture process, the counting method and the cell counter used were the same as those in Example 1, ensuring that the cell density did not exceed 8*10\hat{ }5/ml. If the cells were too dense, dividing those in the wells in time and adding fresh medium.

After treating cells with small molecule inhibitors for 7 days, the expression of LT-HSCs cell surface markers (CD34+CD45+CD90+CD45RA−CD38−) was detected by flow cytometry using the same method as in Example 3.

Mice were provided, with 8 mice per group. Mice were purchased from Beijing Weitongda Biotechnology Co., Ltd., the strain was NPG (NOD-Prkdcscidll2rgnull/Vst), 6-week-old female mice, and the weight difference between mice was controlled within 3 g. The mice were irradiated with a half-lethal dose before cell transplantation, and the irradiation dose was 1.6 Gy.

The cultured cell suspension (the initial number of culture cells was 0.28*10\hat{ }5/ml/well) was collected, centrifuging at room temperature, centrifuging at 400 g for 5 min, discarding the supernatant, resuspending the cell pellet with 100 μl of physiological saline (containing 1% HSA) and mixing well. Then, an irradiated NPG mouse was injected through the tail vein, and different groups of mice were labeled.

After the cells were transplanted into the mice, the mice were sacrificed at the 18th week, and the bone marrow cells of the mice were collected. The proportions of human CD45, human CD19, human CD3, human CD33 and human CD56 were detected by flow cytometry. Antibodies, 7-AAD dyes and sources used in this example were shown in Table 5.

TABLE 5
Antibodies and 7-AAD Dye

| Name of antibody | manufacturer | Cat. No. |
|---|---|---|
| FITC anti-mouse CD45 | Biolegend | 103108 |
| APC/Cy7 anti-human CD45 | Biolegend | 304014 |
| Brilliant Violet 510 ™ anti-human CD3 | Biolegend | 300448 |
| PE anti-human CD19 | Biolegend | 363004 |
| Brilliant Violet 421 ™ anti-human CD33 | Biolegend | 303416 |
| APC anti-human CD56 | Biolegend | 304610 |
| 7-AAD Viability Staining Solution | Biolegend | 420404 |

The proportions of human CD45, human CD19, human CD3, human CD33 and human CD56 in mouse bone marrow cells were detected by flow cytometry. The set groups for cell detection were shown in Table 6.

TABLE 6

| | Cell | | Antibody |
|---|---|---|---|
| Group | number | Name of added antibody | amount |
| NC | 2 × 10^{∧}5 | — | — |
| Sample | 2 × 10^{∧}5 | FITC anti-mouse CD45 | 2 μL |
| | | APC/Cy7 anti-human CD45 | 2 μL |
| | | Brilliant Violet 510 ™ anti-human CD3 | 2 μL |
| | | PE anti-human CD19 | 2 μL |
| | | Brilliant Violet 421 ™ anti-human CD33 | 2 μL |
| | | APC anti-human CD56 | 2 μL |

The mice were sacrificed by cervical dislocation, and the tibia and femur of one hind leg of the mice were taken. Ophthalmic scissors and ophthalmic forceps were used to cut off both ends of the tibia and femur respectively to expose the bone marrow cavity. A 1 ml syringe was used to suck pre-cooled PBS (containing 1% HSA), the needle was inserted into one end of the bone marrow cavity, and the PBS was injected forcefully to flush out the bone marrow cells from the other end of the bone marrow cavity. Tibial and femoral bone marrow cavities were rinsed with 2 ml of PBS, respectively. The bone marrow cell suspension was repeatedly blew and sucked with a pipette, filtered the cell suspension with a 40 um cell mesh (BD, Cat. number: 352340), and centrifuged at room temperature at 400 g for 5 min. After centrifugation, the supernatant was discarded, and the bone marrow cells were used for later use.

1 ml of erythrocyte lysate was added to the spare bone marrow cells, mixing by vortexing, and lysing at room temperature for 15 minutes. During this period, the samples were mixed upside down every 3 minutes. After the lysis, 4 ml of PBS (containing 1% HSA) was added to each sample, and centrifuged at room temperature at 400 g for 5 min After centrifugation, the supernatant was discarded, adding 1 ml of PBS (containing 1% HSA) to each sample, and mixing by vortexing. 100 μl of cell suspension was taken from each sample, adding antibodies according to the groups in Table 6, mixing by vortexing, and incubating at room temperature for 15 min in the dark. After the incubation, 5 μl of 7-AAD dye was added to each sample group, mixed by vortexing, and incubating at room temperature for 5 min in the dark. After the incubation, 1 ml of PBS (containing 1% HSA) was added to the NC and each sample group, mixing well, centrifuging at room temperature at 400 g for 5 min After centrifugation, the supernatant was discarded, 100 μl of PBS (containing 1% HSA) was added to each experimental sample to resuspend the cells, and flow cytometry was used for detection.

The test results were analyzed as follows: 1) The target cell population was human CD45+ cell population, human CD19+ cell population, human CD3+ cell population, human CD33+ cell population, and human CD56+ cell population; 2) The determination of the logic gate and gate position was shown in FIG. 7: First delineating the cell population, P1 gate; removing the adherent cells from the cell population derived from the P1 gate, as the P2 gate; delineating live cells from cell population derived from the P2 gate with 7-AAD negative cells as P3 gate; delineating mouse CD45+(P4 gate) and human CD45+ cell populations (P5 gate) from cell populations from the P3 gate with NC; delineating human CD33+(P11 gate) and human CD56+ cell populations (PI3 gate) from cell populations derived from the P5 gate with NC; delineating human CD19+ (10 gate) and human CD3+ cell populations (P12 gate) from cell populations derived from the P5 gate with NC. The transplantation efficiency of human hematopoietic stem cells was shown by the proportion of human CD45 cells, and the calculation method was human CD45%/(human CD45%+mouse CD45%). The efficiency of human hematopoietic stem cells in differentiation into each lineage in mice was shown as human CD19% (representing B cells), human CD3% (representing T cells), human CD33% (representing myeloid cells), human CD56% (representing NK cells)), and the results were shown in FIGS. 8A and 8B.

The results of FIG. 8A showed that, under the condition of the same initial number of culture cells for mouse transplantation, the bone marrow transplantation efficiency of the hematopoietic stem cells treated with AG1296 at week 18 was significantly higher than that of the Mock group and the SR1 group. The results of FIG. 8B showed that the proportion of cells of each lineage formed by the differentiation of hematopoietic stem cells treated with AG1296 was not significantly different from the Mock group and the SR1 group, and the ability of hematopoietic stem cells treated with AG1296 to differentiate into cells of each lineage was normal.

The above are only preferred embodiments of the present application, and are not intended to limit the present application in other forms. Any person skilled in the art may use the technical content disclosed above to make changes or modifications to gain equivalent embodiments with equivalent changes. However, without departing from the content of the technical solutions of the present application, any simple modifications, equivalent changes and modifications made to the above embodiments according to the technical essence of the present application still belong to the protection scope of the technical solutions of the present application.

1. A culture medium composition for expanding hematopoietic stem cells (HSCs) and maintaining self-renewal ability and differentiation potential of HSCs, comprising a hematopoietic stem cell medium and a small molecule inhibitor of a PDGFR target.

2. The culture medium composition according to claim 1, wherein the small molecule inhibitor of a PDGFR target is one or more selected from the group consisting of: AG1296, PDGFR inhibitor 1, imatinib, PP121, ponatinib, axitinib, trapidil and erdafitinib.

3. The culture medium composition according to claim 1, wherein the hematopoietic stem cell medium comprises: 1) a basal medium (preferably a serum-free basal medium); 2) a growth factor; or 3) a cytokine.

4. The culture medium composition according to claim 3, wherein the growth factor or cytokine is one or more selected from the group consisting of: growth factor Flt-3L, growth factor SCF, growth factor TPO and interleukin IL-6.

5-6. (canceled)

7. The culture medium composition according to claim 1, wherein the HSCs are derived from bone marrow, mobilized peripheral blood, umbilical cord blood, cryopreserved and resuscitated HSCs or HSCs modified by gene editing.

8. A method for promoting the expansion of HSCs and maintaining the self-renewal capacity of the HSCs, comprising in vitro culturing the HSCs in a culture medium composition containing a small molecule inhibitor of a PDGFR target.

9. The method according to claim 8, wherein the small molecule inhibitor of a PDGFR target is one or more selected from the group consisting of: AG1296, PDGFR inhibitor 1, imatinib, PP121, ponatinib, axitinib, trapidil, and erdafitinib.

10. The method according to claim 8, wherein the hematopoietic stem cell medium comprises: 1) a basal medium; 2) a growth factor; or 3) a cytokine.

11. The method according to claim 10, wherein the growth factor or cytokine is one or more selected from the group consisting of: growth factor Flt-3L, growth factor SCF, growth factor TPO, and interleukin IL-6.

12-13. (canceled)

14. The method according to claim 8, wherein the HSCs are derived from bone marrow, mobilized peripheral blood, umbilical cord blood, cryopreserved and resuscitated HSCs or HSCs modified by gene editing.

15. The method according to claim 8, wherein the in vitro culture time is about 4-21 days.

16. The method according to claim 8, wherein after the in vitro culture, the cell number of CD34+ phenotype HSCs accounts for 40-85% of the total cells.

17. The method according to claim 8, wherein after in vitro culture, the cell number of CD34+CD90+ phenotype HSCs accounts for 6-15% of the total cells.

18. The method according to claim 8, wherein after in vitro culture, the cell number of CD34+CD90+CD45RA− phenotype HSCs accounts for 2-10% of the total cells.

19. The method according to claim 8, wherein after in vitro culture, the cell number of CD34+CD45+CD90+CD45RA−CD38-phenotype HSCs accounts for 2-5% of the total cells.

20. An HSCs infusion solution, wherein the cell number of CD34+ phenotype HSCs accounts for 40-85% of the total cells.

21. The HSCs infusion solution according to claim 20, wherein the cell number of CD34+CD90+ phenotype HSCs accounts for 6-15% of the total cells.

22. (canceled)

23. The HSCs infusion solution according to claim 20, wherein the cell number of CD34+CD45+CD90+CD45RA−CD38− phenotype HSCs accounts for 2-5% of the total cells.

24. (canceled)

25. A method for replenishing blood cells to an individual in need, comprising infusing the HSCs infusion solution of claim 20 to the individual.

26-28. (canceled)

29. A method for preventing or treating a disease in an individual, comprising infusing the HSCs infusion solution of claim 20 to the individual.

30-31. (canceled)
